(19) United States
(12) Patent Application Publication
(10) Pub. No.: US 2010/0203014 A1
(43) Pub. Date: Aug. 12, 2010

(54) ZWITTERIONIC BUFFERED ACIDIC PEPTIDE AND PROTEIN FORMULATIONS

(75) Inventor: Edward T. Maggio, San Diego, CA (US)

Correspondence Address: DLA PIPER LLP (US), 4365 EXECUTIVE DRIVE, SUITE 1100, SAN DIEGO, CA, 92121-2133, US

(73) Assignee: Aegis Therapeutics LLC, San Diego, CA (US)

(21) Appl. No.: 12/699,776

(22) Filed: Feb. 3, 2010

Related U.S. Application Data
(60) Provisional application No. 61/149,882, filed on Feb. 4, 2009.

(51) Int. Cl. A61K 38/21; A61K 38/02; A61K 38/08; A61K 38/26; A61K 38/23; A61K 38/28; A61K 38/22; A61K 38/27; A61P 3/10

(52) U.S. Cl. 424/85.4; 514/2; 514/16; 514/12; 514/3; 514/8

(57) ABSTRACT

The present invention provides glycine buffered pharmaceutical compositions suitable for nasal administration and methods for nasal delivery of such compositions. The pharmaceutical compositions described herein include a therapeutically active peptide and/or protein admixed with an aqueous solution buffered with glycine and having a pH in the ranging from about 3.0 to 4.5. The compositions are formulated to maintain stability of the peptide for an extended period of time, but do not significantly change the normal pH of nasal mucosal secretions avoiding irritation and burning sensation in the nose upon administration.

### CROSS REFERENCE TO RELATED APPLICATION(S)

This application claims the benefit of priority under 35 U.S.C. §119(e) of U.S. Ser. No. 61/149,882, filed Feb. 4, 2009, the entire content of which is incorporated herein by reference.

### BACKGROUND OF THE INVENTION

1. Field of the Invention

The present invention relates generally to pharmaceutical compositions and more specifically to pH stabilized pharmaceutical compositions including therapeutically active peptides formulated for nasal administration.

2. Background Information

Intranasal administration of peptide and protein drugs has been found to be a useful drug delivery route for this class of compounds. The pH at which peptides and proteins are optimally stabilized, however, is significantly acidic and different than the normal pH of the nasal mucosal. Unfortunately, solutions having a pH in the acidic range can have detrimental effects on the interior of the nose. Accordingly, there is an inherent conflict in selecting an appropriate pH for peptide formulation stability and nasal comfort.

Drug formulations having pH values that differ substantially in pH and osmolarity from the normal values can result in irritation of the nasal cavity. In particular, solutions having a pH in the acidic range can cause a burning sensation in the nose.

Buffer components typically used for intranasal administration include the acetic acid/acetate and lactic acid/lactate systems. When formulated at pH values in the pH 4 to pH 7 range, these buffer systems are substantially unprotonated and the resulting ionic forms are not volatile and therefore do not impart a smell or taste to the drug formulation. However, at more acidic pH values, the free acid species, namely acetic acid and lactic acid, are increased. Since the free acids are volatile they impart a vinegar-like or sour milk-like smell and taste respectively. The smell increases as the pH is lowered. Below the pKa of acetic acid (pKa=4.74) and lactic acid (pKa=3.86) their respective volatile free acid species predominate and the undesirable smell is significantly increased.

In general, the normal pH found in the nasal mucosa is approximately pH 5.5 to pH 6.5. For example, it has been determined that the average pH in the anterior of the human nose is about 6.40 (+0.11, −0.15 S.D.) and the pH in the posterior of the nasal cavity is 6.27 (+0.13, −0.18 S.D.). However, it is also established that an optimal pH range for stabilization of peptides and/or proteins is between about pH 3.5 and pH 4.5. Thus the difficulty of providing protein stability while avoiding nasal irritation upon administration is clear.

Typically, the proteins found in normal nasal secretions including albumin, immunoglobulins, lysozyme, and other enzymes contain carboxylic groups and amino groups that provide a buffer capacity that helps to keep the nasal pH in its normal range. Intranasal administration of a buffered solution having an acidic low pH, for example in the pH range of about 3.5 to 4.5, so as to stabilize peptide drugs, can move the pH found in nasal secretions from the normal level, approximately pH 6, down into the more acidic range resulting in detrimental side effects to the subject including irritation to the nasal mucosa and the nasopharynx, burning sensation in the nose or throat (rhinitis or pharyngitis), rhinorhea, lacrimation, sneezing, nasal congention, or bad taste or smell.

It has been determined that administration of acidic buffers into the nasal cavity triggered a reflex increase in mucus production in response to a perceived irritant. In addition to being irritating to a patient's mucosal tissue, the increased mucous secretion upon nasal administration of acidic buffers inhibits drug absorption through the nasal mucosa through dilution and enhanced mucociliary clearance and may cause the patient to sneeze or otherwise expel the excess mucous along with drug and drug formulation excipients further resulting in decreased drug absorption.

In order to maintain a specific desired pH, buffer compounds typically, carboxylic acids or amines, are added to a drug formulation. Typical physiologically compatible buffer compounds used in pharmaceutical compositions include acetate, citrate, and phosphate. Buffers including acetate, citrate, or phosphate, exert strong buffering capacity in a pH range from pH 2 to pH 9 and beyond. In particular, the polyvalent anionic buffers phosphate and citrate offer strong buffering capacity across a very broad pH range, since the pKa's of the multiple dissociable groups span a broad pH range. For example, citric acid (citrate) has three pKa's 3.15, 4.77 and 6.40; phosphate pKa's are 2.15, 7.2 and 12.4; and the pKa of acetate is 4.76. Of particular relevance to inclusion in nasal formulations, they confer strong buffer capacity in the pH 3 to pH 7 range, even at low concentrations in the 100 μM or lower range. Since a principle function of the buffer compounds present in the aqueous drug formulations is to resist changes in pH, intranasal administration of a peptide or protein formulation buffered at about pH 3.5 to pH 4.5 incorporating one of the buffers cited above can result in acidification or reduction of the nasal mucosal pH resulting one or more of the detrimental side effects discussed above.

Accordingly, pharmaceutical compositions suitable for nasal delivery of therapeutically active peptides and/or proteins using certain buffer formulations which confer both peptide stabilization as well as avoidance of irritation to the nasal cavity upon delivery of the composition to the nasal mucosa are desirable. For example, pharmaceutical compositions including a buffer and capable of maintaining a pH in the range of from about pH 3.5 to 4.5 so as to stabilize peptide drugs, but which, when administered intranasally, do not significantly perturb the normal pH of the nasal mucosa are desirable for non-irritating nasal delivery applications of pH stabilized therapeutically active peptides and/or proteins.

### SUMMARY OF THE INVENTION

present invention is based in part, on the discovery that certain zwitterionic compounds, in particular naturally occurring neutral non-polar amino acids that are biologically compatible, stable upon storage in aqueous media, and chemically and photochemically non-reactive, can be used to make non-toxic, non-irritating aqueous buffered compositions that maintain a low pH level in the pH 3 to pH 4.5 range required for optimal peptide or protein stabilization during storage while providing only minimal disruption of the normal and near neutral pH of the nasal mucosa upon nasal administration. Similar considerations apply to subcutaneous, intramuscular, or intravenous injection of acidic peptide solutions into the normal and near neutral pH of other sensitive tissues.

In one embodiment, the present invention provides a method for nasal administration of an acidic pharmaceutical composition including a therapeutically active peptide and/or protein to a subject. The method includes administering to the nasal mucosa of the subject a pharmaceutical composition which includes a therapeutically active peptide and an aqueous buffer solution containing a zwitterionic non-polar amino acid. The composition may further include an absorption enhancing excipient. The composition is buffered to a pH of about 3.0 to 4.5. In various aspects, the pH of the nasal mucosa after administration remains essentially unchanged or maintained within 1 pH unit or less of the pH before administration of the pharmaceutical composition. In an exemplary aspect, the amino acid is glycine.

Accordingly, in another embodiment, the present invention provides acidic buffered pharmaceutical compositions suitable for nasal administration of a therapeutically active peptide and/or protein that do not cause unacceptable nasal irritation. The pharmaceutical compositions include a therapeutically active peptide and/or protein and an aqueous solution including at least one non-polar zwitterionic amino acid, such as, glycine, alanine, valine, leucine, isoleucine and phenylalanine, buffered to a pH of about 3.0 to 4.5. The composition may further include other excipients such as stabilizers or absorption enhancers.

The compositions are buffered to a pH of about 3.0 to 4.5 and suitable for nasal administration. In various aspects, the therapeutically active peptide may be peptides or analogs such as, but not limited to, octreotide, glucagon, calcitonin, insulin, amylin, beta interferon, gamma interferon, alpha interferon, erythropoietin, growth hormone releasing hormone, human growth hormone, parathyroid hormone, glucagon-like peptide-1, exendin-4, liraglutide, taspoglutide, pramlintide, leptin, metreleptin (methionyl recombinant leptin; r-metHuLeptin), D-ala-Peptide-T-amide (DAPTA), OB3, [D-Leu-4]-OB3, peptide YY (PYY), growth colony stimulating factor (GCSF), C-peptide of insulin, or analogs and derivatives thereof. Additionally, the compositions may include other agents, such as, alkylsaccharides, preservatives, and osmolarity adjusting agents. In various aspects, the compositions retain greater than 93% potency after storage at 25° C. for 12 months or 87% potency after storage at 40° C. for 12 months.

### BRIEF DESCRIPTION OF THE DRAWINGS

FIG. 1 is a graphical representation of glucagon purity. The graphic shows the percentage of initial glucagon purity over 12 months as determined by RP-HPLC maintained at 5, 15, and 25° C., pH 3.5, 10 mg/mL glucagon in aqueous 30 mM glycine buffer solution with 0.125% dodecyl-beta-D-maltoside as antimicrobial preservative.

### DETAILED DESCRIPTION OF THE INVENTION

The present invention is based in part, on the discovery that certain zwitterionic compounds, in particular naturally occurring neutral non-polar amino acids that are biologically compatible, stable upon storage in aqueous media, and chemically and photochemically non-reactive, can be used to make non-toxic, non-irritating aqueous buffered compositions that maintain a low pH level in the pH 3 to pH 4.5 range required for optimal peptide or protein stabilization during storage while providing only minimal disruption of the normal and near neutral pH of the nasal mucosa upon nasal administration.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

One means of stabilizing peptide and/or protein therapeutics prior to administration to the patient is to lyophilize or freeze-dry the peptide or protein, and then reconstitute immediately prior to administration. This circumvents problems associated with long-term storage of aqueous solutions of the peptide or protein drug at pH values that are unsuitable for the route of administration. An example is glucagon, which is administered as a rescue therapy for diabetics undergoing hypoglycemia. Unfortunately, diabetic patients experiencing hypoglycemia may be unable to conduct the necessary manipulations of reconstituting lyophilized glucagon ensuring its dissolution, drawing it into a syringe and administering it by injection. These actions are relatively complicated and take a considerable period of time. Since time is of the essence in treating hypoglycemia and since the diabetic's physical capabilities may be compromised by severe hypoglycemia, these types of manipulations are difficult, sometimes impractical, and in severe cases of hypoglycemia, inability to undertake them in a timely manner may lead to serious complications including death. The compositions of the present invention have utility as formulations of ready-to-use nasally administered peptide and/or protein therapeutics that exhibit sufficient stability to be commercially viable, up to one year or more at refrigerated temperatures, and that do not cause nasal irritation as a result of acidification of the nasal mucosa by buffering components present in the compositions.

Accordingly, the present invention provides improved pharmaceutical compositions which exhibit exceptional characteristics for nasal delivery of therapeutic peptides and/or proteins and methods of nasal delivery using such compositions. As used herein, the terms “peptide” and “protein” are intended to be synonymous, and used broadly to refer to macromolecules comprising linear polymers of amino acids which may act in biological systems, for example, as structural components, enzymes, chemical messengers, receptors, ligands, regulators, hormones, and the like.

Therapeutic peptides, whether naturally or synthetically derived, for use with the present invention may be any medically or diagnostically useful peptide. The peptides may be of various sizes including up to about 1 kD, 15 kD, 30 kD, 40 kD, 50 kD, 60 kD, 70 kD, 80 kD, 90 kD, 100 kD, 165 kD, and larger. By way of example, and in no way limiting, peptides for use with the present invention include vasopressin, vasopressin polypeptide analogs, desmopressin, glucagon, corticotropin (ACTH), gonadotropin, calcitonin, C-peptide of insulin, parathyroid hormone (PTH), PTH derivatives such as PTH 1-34 and PTH 1-31, growth hormone (HG), human growth hormone (hGH), growth hormone releasing hormone (GHRH), oxytocin, corticotropin releasing hormone (CRH), somatostatin or somatostatin polypeptide analogs, gonadotropin agonist or gonadotrophin agonist polypeptide analogs, human atrial natriuretic peptide (ANP), human thyroxine releasing hormone (TRH), follicle stimulating hormone (FSH), prolactin, insulin, insulin like growth factor-I (IGF-I) somatomedin-C (SM-C), calcitonin, leptin, beta interferon, gamma interferon, alpha interferon, OB3, [D-Leu-4]-OB3, melatonin, glucagon-like peptide-1 (GLP-1), GiP, neuropeptide pituitary adenylate cyclase, GM-1 ganglioside, nerve growth factor (NGF), nafarelin, D-tryp6)-LHRH, FGF, VEGF antagonists, leuprolide, interferon (e.g., α, β, γ, and the like), low molecular weight heparin, peptide YY (PYY), oxyntomodulin and related peptides such as TKS1225, LHRH antagonists, keratinocyte growth factor (KGF), glial-derived neurotrophic Factor (GDNF), ghrelin, ghrelin antagonists, octreotide, amylin, exendin-4, liraglutide, taspoglutide, pramlintide, growth colony stimulating factor, D-ala-Peptide-T-Amide (DAPTA), monoclonal antibodies, humanized monoclonal antiobodies, antibody-derived fragments, and the like. Further, therapeutic peptides are intended to include analogs (e.g., similar structure) and derivatives (e.g., amino acid substitutions, mutations, deletions or trucations, additions, and the like) thereof.

As used herein, an “interferon” is inteded to include commonly used forms of intereron, such as α, β, and γ interferon, as well as derivatives and analogs thereof. For example, an interferon may include interferon alpha 2a, interferon alpha 2b, Human leukocyte Interferon-alpha (HuIFN-alpha-Le), interferon beta 1a, interferon beta 1b, pegylated interferon alpha 2a, and pegylated interferon alpha 2b.

It was discovered that the amino acid glycine is effective in buffering therapeutic peptide drug formulations in the pH range of from about 3.0 to 4.5, but does not significantly modify the normal pH of nasal mucosal secretions when used at low concentrations. In particular, it has been found that aqueous compositions comprising peptides or proteins, and glycine or one or more other neutral non-polar aminoacids as a buffering agent, when prepared having a pH in the range of 3.5-4.5, maintain stability for an extended period of time, but do not significantly change the normal pH of nasal mucosal secretions. It was further discovered that unlike acetate or lactate, acidicly buffered solutions of glycine and other neutral non-polar do not present any smell or taste sensation when used as described herein and are non-reactive with many commonly used excipients. Thus the present invention provides compositions suitable for nasal delivery of stabilized therapeutic peptides having an acidic pH while avoiding such detrimental side effects to the subject as irritation to the nasal mucosa and the nasopharynx, burning sensation in the nose or throat (rhinitis or pharyngitis), rhinorhea, lacrimation, sneezing, nasal congention, or bad taste or smell.

As discussed herein, buffer components may also contribute to unwanted smell or, due to normal mucociliary clearance from the nose into the throat, unwanted taste. For example, acetate-based buffers or lactate-based buffers at low pH contain significant amounts of the corresponding protonated species, acetic acid and lactic acid. While the unprotonated or negatively charged acetate or lactate species are essentially free of smell, the protonated (uncharged or free acid) species impart a vinegar-like or sour-milk-like smell, respectively. It was discovered that zwitterionic buffers do not impart a smell or unpleasant taste at acidic pH values. Examples of zwitterionic buffer components useful for intranasal administration of the drug formulation stabilized by acidic buffer conditions described herein, are the non-polar amino acids, such as, glycine, alanine, valine, leucine, isoleucine and phenylalanine. These particular substances are naturally occurring, very stable upon aqueous storage, and are non-toxic, and unlike other amino acids, are not reactive with proteins through disulfide bond formation or disulfide exchange, transesterification, oxidation, or photoreactivity. They have the further advantage of being designated GRAS (Generally Recognized as Safe) substances, making them desirable for use as excipients in human therapeutics.

Additionally, with regard to the non-polar amino acid glycine, a particularly attractive feature of glycine is that it has a natural sweet taste so that as it is cleared from the nasal passages to the back of the oral cavity via the normal mucociliary clearance process, it does not impart an unpleasant taste. As discussed further below, since artificial and natural sweeteners are often added to drug formulations to increase palatability or mask unpleasant drug taste, the natural sweet taste of glycine makes it an exemplary buffer system.

Accordingly, in one embodiment, the present invention provides a method for nasal administration of an acidic pharmaceutical composition including a therapeutically active peptide and/or protein to a subject. The method includes administering to the nasal mucosa of the subject a pharmaceutical composition which includes a therapeutically active peptide, an aqueous solution buffered with glycine, and an absorption enhancing amount of an alkyl saccharide.

An exemplary characteristic of the glycine buffered pharmaceutical compositions of the present invention is that upon nasal administration of the composition is relatively unperturbed. In various aspects, the pH of the nasal mucosa after administration remains unchanged or maintained within 1 pH point or less of the pH before administration of the pharmaceutical composition. Accordingly, the pH of the nasal mucosa after administration is maintained within less than 1, 0.9, 0.8, 0.7, 0.6, 0.5, 0.4, 0.3, 0.2 or 0.1 pH points of the pH before administration of the pharmaceutical composition.

In another embodiment, the invention provides buffered pharmaceutical compositions suitable for nasal administration of a therapeutically active peptide and/or protein. The pharmaceutical compositions include a therapeutically active peptide and/or protein and an aqueous solution including glycine.

In various aspects, the glycine buffered compositions are prepared to have an acidic pH, in part to maintain stability and prevent degradation of the peptide for an extended period of time. In one aspect, the composition has a pH of about 3.0 to 4.5. Accordingly, the acidic pH of the composition may be in the range of about 3.0 to 3.5, 3.5 to 4.0, or 4.0 to 4.5. Alternatively, the acidic pH of the composition may be about 3.0, 3.1, 3.2, 3.3, 3.4, 3.5, 3.6, 3.7, 3.8, 3.9, 4.0, 4.1, 4.2, 4.3, 4.4 or 4.5.

In various aspects, the compositions of the present invention may be formulated to include additional agents. For example, the compositions may be formulated to include one or more alkylsaccharides. As used herein, “alkylsaccharide” refers to any sugar joined by a linkage to any hydrophobic alkyl, as is known in the art. Preferably the alkylsaccharide is nonionic as well as nontoxic. Alkylsaccharides are available from a number of commercial sources and may be natural or synthesized by known procedures, such as chemically or enzymatically.

In various aspects, alkylsaccharides of the present invention may include, but are not limited to alkylsaccharides, such as octyl-, nonyl-, decyl-, undecyl-, dodecyl-, tridecyl-, tetradecyl-, pentadecyl-, hexadecyl-, heptadecyl-, and octadecyl-α- or β-D-maltoside, -glucoside or -sucroside; alkyl thiomaltosides, such as heptyl, octyl, dodecyl-, tridecyl-, and tetradecyl-β-D-thiomaltoside; alkyl thioglucosides, such as heptyl- or octyl 1-thio α- or β-D-glucopyranoside; alkyl thiosucroses; alkyl maltotriosides; long chain aliphatic carbonic acid amides of sucrose β-amino-alkyl ethers; derivatives of palatinose and isomaltamine linked by amide linkage to an alkyl chain; derivatives of isomaltamine linked by urea to an alkyl chain; long chain aliphatic carbonic acid ureides of sucrose β-amino-alkyl ethers; and long chain aliphatic carbonic acid amides of sucrose β-amino-alkyl ethers.

As described above, the hydrophobic alkyl can thus be chosen of any desired size, depending on the hydrophobicity desired and the hydrophilicity of the saccharide moiety. For example, the range of alkyl chains for use with the present invention is from about 9 to about 24 carbon atoms. Another range for use with the present invention is from about 10 to about 14 carbon atoms. Similarly, some preferred alkylglycosides include maltose, sucrose, and glucose linked by glycosidic or ester linkage to an alkyl chain of 9, 10, 12, 13, 14, 16, 18, 20, 22, or 24 carbon atoms, e.g., nonyl-, decyl-, dodecyl- and tetradecyl sucroside, glucoside, maltoside, and the like. These compositions are nontoxic, since they are degraded to an alcohol or fatty acid and a saccharide, and are amphipathic. Additionally, the linkage between the hydrophobic alkyl group and the hydrophilic saccharide can include, among other possibilities, a glycosidic, thioglycosidic, amide, ureide, or ester linkage.

As use herein, a “saccharide” is inclusive of monosaccharides, oligosaccharides or polysaccharides in straight chain or ring forms, or a combination thereof to form a saccharide chain. Oligosaccharides are saccharides having two or more monosaccharide residues.

Accordingly, examples of saccharides include glucose, maltose, maltotriose, maltotetraose, sucrose and trehalose.

In sugar chemistry, an anomer is either of a pair of cyclic stereoisomers (designated α or β) of a sugar or saccharide, differing only in configuration at the hemiacetal (or hemiketal) carbon, also called the anomeric carbon or reducing carbon. If the structure is analogous to one with the hydroxyl group on the anomeric carbon in the axial position of glucose, then the sugar is an alpha anomer. If, however, that hydroxyl is equatorial, the sugar is a beta anomer. For example, α-D-glucopyranose and β-D-glucopyranose, the two cyclic forms of glucose, are anomers. Likewise, alkylsaccharides occur as anomers. For example, dodecyl β-D-maltoside and dodecyl α-D-maltoside are two cyclic forms of dodecyl maltoside. The two different anomers are two distinct chemical structures, and thus have different physical and chemical properties. In one aspect of the invention, the alkylsaccharide of the present invention is a β anomer. In an exemplary aspect, the alkylsaccharide is a β anomer of an alkylmaltoside, such as dodecyl β-D-maltoside. In various aspects the alkylsaccharide for use with the present invention is selected from the group consisting of dodecyl sucrose, dodecyl maltoside, tridecyl maltoside, tetradecyl maltoside, sucrose monododecanoate, sucrose monotridecanoate, sucrose monotetradecanoate, sucrose monostearate, sucrose distearate, or a combination thereof.

The compositions of the present invention may also include one or more osmolarity adjusting agents to obtain the desired final osmolarity. Examples of suitable osmolarity adjusting agents include, but are not limited to sodium and potassium chloride; monosaccharides, such as dextrose; sugar alcohols, such as mannitol; calcium and magnesium chloride; and low molecular weight polyols, such as glycerin and propylene glycol. Typically, these agents are used individually in amounts ranging from about 0.01 to 5 weight percent and preferably, from about 0.1 to about 2 weight percent. In exemplary aspects, the osmolarity adjusting agent is mannitol, sodium chloride, potassium chloride, or combinations thereof.

The compositions of the present invention may also include additional agents, such as preservatives. While any suitable preservative is envisioned for use with the present invention, one such example is ethylene-diamine-tetra-acetic acid (EDTA) including its corresponding sodium or potassium salts.

Pharmaceutically acceptable carriers useful for formulating a composition for administration to a subject are well known in the art. Such carriers include, for example, aqueous solutions such as water. As used herein, an “aqueous solution” is intended to mean a solution which incorporates water as solvent. In various aspects, the compositions of the present invention include aqueous solutions buffered only with glycine, with water as the only solvent. However, one of skill in the art would understand that further additional agents may be added to the compositions of the present invention to optimize characteristics of the composition for nasal administration depending on the physicochemical characteristics of the therapeutic peptide. For example the compositions may include additional polymers, such as natural gums, hydroxy propyl methyl cellulose, modified starches, alginates, and carboxy vinyl copolymer; carbohydrates, such as glucose, sucrose or dextrans; antioxidants, such as ascorbic acid or glutathione, ascorbic acid ethyl ester, ascorbyl palmitate, butylated hydroxy anisole, butylated hydroxy toulene, propylene glycol, polypropylene glycols, and tocopherol or its derivatives; chelating agents; stabilizers; excipients; anti-microbial agents; coloring agents; and flavorings. Additionally, while an exemplary aspect the compositions include only glycine as a buffering agent, additional buffering agents known in the art may be used in addition to glycine such as, but not limited to, the other non-polar amino acids cited above.

As discussed herein, compositions of the present invention for intranasal administration may contain other substances or excipients in addition to a buffer. Such excipients may include, for example, stabilizers; taste masking compounds such as sweeteners or flavoring agents; cosolvents such as propylene glycol, polypropylene glycols of different lengths, and ethanol; osmolarity adjusting agents such as sodium chloride and mannitol; absorption enhancers such as alkyl saccharides, chitosan, and beta methyl cyclodextrin; and preservatives such as benzalkonium chloride and phenyl ethyl alcohol. It is therefore important that the buffering agent not be reactive with such materials. Non-polar amino acids, such as glycine, alanine, valine, leucine, isoleucine and phenylalanine are essentially chemically inert with respect to these excipients.

As discussed further in the Examples included herein, the pharmaceutical compositions of the present invention exhibit prolonged shelf life and confer prolonged stability to peptide therapeutics. It has been determined that the compositions allow for prolonged stability of peptides and therefore prolong the potency of peptides by preventing degradation. Therapeutic peptides incorporated in the compositions described herein, which have been stored for 12 months, retain greater than approximately 97% potency after storage at 5° C., greater than approximately 95% potency after storage at 15° C., greater than approximately 85% potency after storage at 25° C. as shown in Example 3 below.

As described herein, therapeutic peptides formulated in acidic glycine buffered compositions are suitable for administration to a subject. The terms “administration” or “administering” as used herein are defined to include the act of providing a pharmaceutical composition of the invention to a subject in need of treatment. While the compositions described herein may be suitable for administration via any well known route, an exemplary administration route is nasal or intranasal administration. As used herein, the terms “nasal” and “intranasal” administration are synonymous and intended to include administration to mucosal tissue lining the nasal cavity and the epithelial linings of the airway (e.g., trachea, bronchus, bronchioles and the like). Accordingly, in an exemplary aspect, the pharmaceutical compositions of the present invention, are formulated into pharmaceutically acceptable forms suitable for nasal administration, such as sprays.

The term “subject” as used herein refers to any individual or patient to which the subject methods are performed. Generally the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject.

The total amount of a peptide to be administered in practicing a method of the invention can be administered to a subject as a single dose or application (e.g., a single nasal application) over a relatively short period of time, or can be administered using a fractionated treatment protocol, in which multiple doses or applications are administered over a prolonged period of time. One skilled in the art would know that the amount of the peptide used to treat a subject depends on many factors such as the ailment or disease being treated, the age and general health of the subject as well as the number of treatments to be administered. In view of these factors, the skilled artisan would adjust the particular dose as necessary. In general, the dosage and frequency of administration are determined, initially, using Phase I and Phase II clinical trials. A suitable daily dose of a therapeutic peptide is generally that amount of the peptide which is the lowest dose effective to produce a therapeutic effect. Such an effective dose will generally depend upon the factors described above.

If desired, the effective daily dose of the therapeutic peptide may be administered as two, three, four, five, six or more sub-doses administered separately at appropriate intervals throughout the day, optionally, in unit dosage forms (e.g., single nasal applications). There may be a period of no administration followed by another regimen of administration.

It will be understood, however, that the specific dose level and frequency of dosage for any particular patient may be varied and will depend upon a variety of factors including the activity of the peptide employed, the metabolic stability and length of action of that compound, the age, body weight, general health, sex, diet, mode and time of administration, rate of excretion, drug combination, the severity of the particular condition, and the host undergoing therapy.

Accordingly, depending on the desired dosage for each application, the concentration of drug may be varied within the composition to allow for an appropriate amount of buffered solution to be delivered such that the pH of the nasal mucosa is unperturbed. Dispensing volumes lower than 50 μl by nasal pump is reported as being unacceptable as dose accuracy and probability of effective delivery to target organs is not assured; on the other hand, a higher volume, over 150 is considered in the art as unsuitable as this is known to lead to flooding. Accordingly, the compositions of the present invention may be administered at a single dose volume of from about 50 to about 150 μl; from about 75 to about 125 μl; from about 80 to about 110 μl; from about 85 to about 100 μl; or about 90 μl. The concentration of the peptide may be adjusted depending on the dose volume and desired amount of peptide to be delivered.

The following examples are provided to further illustrate the advantages and features of the present invention, but are not intended to limit the scope of the invention. While they are typical of those that might be used, other procedures, methodologies, or techniques known to those skilled in the art may alternatively be used.

### Example 1

### Aqueous Formulations of Octreotide

This example illustrates preparation and characterization of aqueous formulations including octreotide. Octreotide is an octapeptide that mimics natural somatostatin pharmacologically, however is a more potent inhibitor than natural somatostatin.

Two developmental aqueous formulations of octreotide were prepared. Formulation A was prepared with a pH of 4.0 and includes 10 mM aqueous glycine buffer solution containing 0.5% mannitol, 1 mg/mL octreotide and 0.125% dodecyl-maltoside. Formulation B was prepared with a pH of 4.5 and includes 10 mM aqueous glycine buffer solution, 0.18% dodecyl-maltoside, and 0.1% disodium EDTA. Each formulation was prepared and filled into glass vials with rubber closures. The formulations were maintained for timed intervals at −20, 5, 25, and 40° C. and tested for appearance, pH, octreotide potency and octreotide percent purity.

The octreotide potency and percent purity determinations were determined by HPLC using the methods described in Kyaterekara et al. (J. Pharm. Biomed. Anal., 21, 1999, 327-330). The HPLC method describe by Kyaterekara et al. allows adequate chromatographic performance and separation of octreotide from its major degradation product, [des-Thr-ol⁸]-octreotide. The reverse-phase HPLC method uses a LiChrospher™-60 RP-select B column with a mobile phase composition of acetonitrile/phosphate buffer (pH 7.4, 20 mM) (35:65 v/v) with UV detection at 210 nm. For this protocol, 60 vials per formulation were maintained for timed intervals at −20, 5, 25, and 40° C. as shown in Table I. Test Articles were selected and tested based on the schedule in Table I below.

TABLE I
Stability Schedule

| Condition, | Number of Samples Pulled at Time point (months) | | | | | | | | | Contingency | Total |
|---|---|---|---|---|---|---|---|---|---|---|---|
| ° C. | 0 | 0.25 | 0.5 | 1 | 1.5 | 2 | 3 | 6 | 12 | Vials | Vials |
| −20 | 1 | 1 | 1 | 1 | 1 | 1 | 1 | 1 | 1 | 11 | 20 |
| 5 | 1 | 0 | 0 | 1 | 0 | 0 | 1 | 1 | 1 | 5 | 10 |
| 25 | 1 | 0 | 0 | 1 | 0 | 0 | 1 | 1 | 1 | 5 | 10 |
| 40 | 2 | 2 | 2 | 2 | 2 | 2 | 2 | 2 | 2 | 2 | 20 |
| Grand Total | | | | | | | | | | 28 | 60 |

Octreotide potency was determined by the ratio of the octreotide peak area to the area obtained for the −20° C. control at each condition and time interval. Similar results may be obtained with glucagon which is typically administered at a 1 mg dose for treating hypoglycemia, but which may be administered in lower doses as an alternative to administration of glucose in cases of mild hypoglycemia. Results are reported as shown in Table II.

TABLE II
Stability of Octreotide Formulation as Assessed by HPLC

| Condition, | Percent Octreotide Purity at each time point (months) | | | | | | | | |
|---|---|---|---|---|---|---|---|---|---|
| ° C. | 0 | 0.25 | 0.5 | 1 | 1.5 | 2 | 3 | 6 | 12 |
| −20 | 100% | 100% | 100% | 100% | 100% | 100% | 100% | 100% | 100% |
| 5 | 100% | 100% | 100% | 100% | 100% | 100% | 100% | 100% | 99% |
| 25 | 100% | 100% | 100% | 100% | 100% | 99% | 100% | 98% | 93% |
| 40 | 100% | 100% | 100% | 99% | 99% | 95% | 96% | 93% | 87% |

### Example 2

### Aqueous Formulations of Octreotide

This example shows the perturbation of nasal mucosal pH during nasal administration of various types of buffered compositions.

Nasal lavage fluid was collected by spraying five successive 100 μL aliquots of sterile water (wfi) for injection into a single nasal cavity and immediately collecting the runoff out of the nasal cavity into a glass test tube held immediately below the naris. Samples from a single volunteer were collected no more frequently than once an hour, which allows for four normal mucociliary clearance half-times of 15 minutes each to allow reestablishment of the normal nasal mucosal secretions. Approximately 300 μL of lavage fluid was collected each time. To 300 μL aliquots of nasal lavage fluid in a glass test tube, was added 90 μL of buffer. All buffers were prepared in sterile water for injection containing 0.5% mannitol and 0.15% dodecyl-maltoside, with and without 0.1% EDTA. After gentle swirling, the pH of the resulting solution is measured. Results are shown in Table III below.

TABLE III
Perturbation of Nasal Mucosal pH by Different Buffers

| | Control | 5 mM glycine | 20 mM glycine | 50 mM glycine | 10 mM phosphate | 10 mM citrate | 10 mM acetate |
|---|---|---|---|---|---|---|---|
| Buffer | (wfi) | buffer, pH 4.0 | buffer, pH 3.8 | buffer, pH 4.5 | buffer, pH 4.0 | buffer, pH 4.0 | buffer, pH 4.0 |
| pH following addition to | 6.1 | 6.0 | 5.4 | 5.8 | 4.5 | 4.6 | 4.5 |
| nasal lavage fluid (with | | | | | | | |
| no EDTA in buffer) | | | | | | | |
| pH following addition to | 6.0 | 5.9 | 5.3 | 5.7 | 4.5 | 4.5 | 4.4 |
| nasal lavage fluid (with | | | | | | | |
| 0.1% EDTA in buffer) | | | | | | | |

### Example 3

### Aqueous Formulations of Glucagon

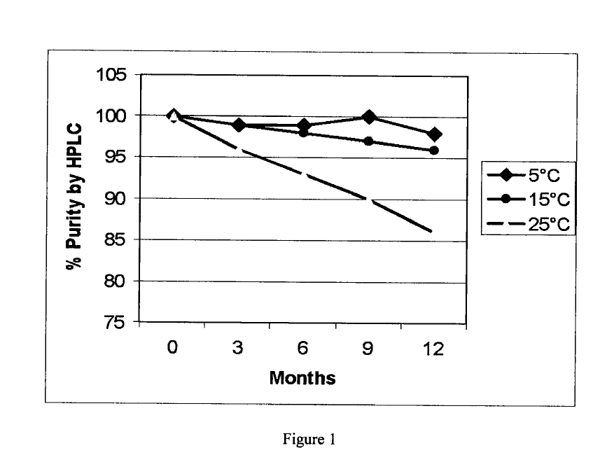

Sterile glucagon injection drug product samples were prepared by aseptic processing at 5 mg/mL and 10 mg/mL in aqueous 30 mM glycine buffer solution, pH 3.0 to pH 4.5 with 0.125% dodecyl-beta-D-maltoside as antimicrobial preservative. The samples were stored in 5-mL glass vials with bromobutyl rubber closures at 5, 15 and 25 degrees C. Glucagon working reference standard solutions were prepared at 7.5 mg/mL in pH 4.0, 30 mM glycine buffer with 0.125% dodecyl-beta-D-maltoside as antimicrobial preservative. Purity was determined by reverse phase (RP) HPLC in 0.1% TFA using a linear gradient of acetonitrile on a Perkin Elmer Series 200 UV/Vis HPLC System™ equipped with an Aquapore™ RP 300 column (4.6×250 mm) at room temperature and at a flow rate of 1 ml/min. The elution was monitored by absorbance at 214 nm. Purity was determined from the ratio of the area under the peak for each sample compared to the area under the peak for the glucagon control stored at −20 deg. C. Examples of the resulting findings are presented in FIG. 1.

Although the invention has been described with reference to the above examples, it will be understood that modifications and variations are encompassed within the spirit and scope of the invention. Accordingly, the invention is limited only by the following claims.

What is claimed is:

1. A method for nasal administration of an acidic pharmaceutical composition to a subject comprising administering to the nasal mucosa of the subject a pharmaceutical composition comprising:
i) a therapeutically active peptide; and
ii) an aqueous solution buffered with a zwitterionic amino acid,
wherein the composition has a pH of about 3.0 to 4.5, thereby administering the acidic pharmaceutical composition to the subject.

2. The method of claim 1, wherein the zwitterionic amino acid is selected from the group consisting of: glycine, alanine, valine, leucine, isoleucine and phenylalanine.

3. The method of claim 2, wherein the zwitterionic amino acid is glycine.

4. The method of claim 1, wherein the pH of the nasal mucosa after administration is maintained within 2 pH units of the pH before administration of the pharmaceutical composition.

5. The method of claim 1, wherein the pH of the nasal mucosa after administration is maintained within 1 pH unit of the pH before administration of the pharmaceutical composition.

6. The method of claim 1, wherein the pH of the nasal mucosa after administration is maintained within 0.5 pH units of the pH before administration of the pharmaceutical composition.

7. The method of claim 1, wherein the therapeutically active peptide is octreotide, glucagon, exendin-4, liraglutide, taspoglutide, pramlintide, leptin, metreleptin, calcitonin, insulin, amylin, pramlintide, an interferon, erythropoietin, growth hormone, human growth hormone, parathyroid hormone, glucagon-like peptide-1, OB3, [D-Leu-4]-OB3, peptide YY (PYY), growth colony stimulating factor, or analogs and derivatives thereof.

8. The method of claim 7, wherein the therapeutically active peptide is octreotide.

9. The method of claim 7, wherein the therapeutically active peptide is glucagon.

10. The method of claim 1, wherein the pharmaceutical composition further comprises an antimicrobial excipient.

11. The method of claim 10, wherein the antimicrobial excipient is an alkylsaccharide.

12. The method of claim 10, wherein the alkylsaccharide has an alkyl chain comprising between about 9 to 24 carbon atoms.

13. The method of claim 10, wherein the alkylsaccharide has an alkyl chain comprising between about 10 to 14 carbon atoms.

14. The method of claim 10, wherein the alkylsaccharide is selected from the group consisting of dodecyl sucrose, dodecyl maltoside, tridecyl maltoside, tetradecyl maltoside, sucrose monododecanoate, sucrose monotridecanoate, sucrose monotetradecanoate, sucrose monostearate, sucrose distearate, or a combination thereof.

15. The method of claim 14, wherein the alkylsaccharide is dodecyl beta-D-maltoside.

16. The method of claim 1, wherein the pharmaceutical composition further comprises ethylene-diamine-tetra-acetic acid (EDTA) or salts thereof.

17. The method of claim 1, wherein the pharmaceutical composition further comprises an osmolarity adjusting agent.

18. The method of claim 17, wherein the osmolarity adjusting agent is mannitol or sodium chloride.

19. The method of claim 10, wherein the pharmaceutical composition retains greater than 80% potency after storage at 25° C. for 12 months.

20. The method of claim 10, wherein the pharmaceutical composition retains greater than 90% potency after storage at 5° C. for 12 months.

21. A pharmaceutical composition comprising:
a) a therapeutically active peptide; and
b) an aqueous solution buffered with a zwitterionic amino acid,
wherein the composition has a pH of about 3.0 to 4.5, and wherein the composition is suitable for nasal administration.

22. The composition of claim 21, wherein the zwitterionic amino acid is selected from the group consisting of: glycine, alanine, valine, leucine, isoleucine and phenylalanine.

23. The composition of claim 22, wherein the zwitterionic amino acid is glycine.

24. The composition of claim 1, wherein the therapeutically active peptide is octreotide, glucagon, exendin-4, liraglutide, taspoglutide, pramlintide, leptin, metreleptin, calcitonin, insulin, amylin, pramlintide, an interferon, erythropoietin, growth hormone, human growth hormone, parathyroid hormone, glucagon-like peptide-1, OB3, [D-Leu-4]-OB3, peptide YY (PYY), growth colony stimulating factor, or analogs and derivatives thereof.

25. The composition of claim 24, wherein the therapeutically active peptide is octreotide.

26. The composition of claim 24, wherein the therapeutically active peptide is glucagon.

27. The composition of claim 21, further comprising an antimicrobial excipient.

28. The composition of claim 21, wherein the antimicrobial excipient is an alkylsaccharide.

29. The composition of claim 28, wherein the alkylsaccharide has an alkyl chain comprising between about 9 to 24 carbon atoms.

30. The composition of claim 28, wherein the alkylsaccharide has an alkyl chain comprising between about 10 to 14 carbon atoms

31. The composition of claim 28, wherein the alkylsaccharide is dodecyl beta-D-maltoside.

32. The composition of claim 28, wherein the alkylsaccharide is selected from the group consisting of dodecyl sucrose, dodecyl maltoside, tridecyl maltoside, tetradecyl maltoside, sucrose monododecanoate, sucrose monotridecanoate, sucrose monotetradecanoate, sucrose monostearate, sucrose distearate, or a combination thereof.

33. The composition of claim 28, further comprising ethylene-diamine-tetra-acetic acid (EDTA) or salts thereof.

34. The composition of claim 28, further comprising an osmolarity adjusting agent.

35. The composition of claim 34, wherein the osmolarity adjusting agent is mannitol or sodium chloride.

36. The composition of claim 28, wherein the pharmaceutical composition retains greater than 90% potency after storage at 5° C. for 12 months.

37. The composition of claim 28, wherein the pharmaceutical composition retains greater than 80% potency after storage at 25° C. for 12 months.
